(19) United States
(12) Patent Application Publication
(10) Pub. No.: US 2026/0335872 A1
(43) Pub. Date: Oct. 29, 2026

(54) CATALYST, PREPARATION METHOD AND APPLICATION FOR CATALYTIC SYNTHESIS OF 2,5-FURANDICARBOXYLIC ACID

(71) Applicant: JINJIANG ANRUN TEXTILE CO., LTD., Quanzhou (CN)

(72) Inventors: Honghui YIN, Zhoukou (CN); Jinbin XIAO, Zhengzhou (CN)

(21) Appl. No.: 19/650,753

(22) Filed: Apr. 17, 2026

(30) Foreign Application Priority Data
Apr. 29, 2025 (CN) 202510554914.0

(51) Int. Cl. B01J 29/04; B01J 6/00; B01J 23/75; B01J 37/00; B01J 37/02; B01J 37/04; C07D 307/54

(52) U.S. Cl. CPC B01J 29/041; B01J 6/001; B01J 6/008; B01J 23/75; B01J 37/0036; B01J 37/0203; B01J 37/0219; B01J 37/0232; B01J 37/04; C07D 307/54; B01J 2229/16

(57) ABSTRACT

Provided are a catalyst, preparation method, and application for catalyzing a synthesis of 2,5-furandicarboxylic acid. The catalyst preparation method includes the following steps: (1) obtaining nitrogen species modified molecular sieve as a catalyst carrier; (2) mixing transition metal salt with the nitrogen modified molecular sieve and subjecting them to high-temperature calcination to obtain a nitrogen modified molecular sieve coated transition metal oxide catalyst. Using 5-hydroxymethylfurfural as raw material, 2,5-furandicarboxylic acid was prepared by catalytic oxidation with this catalyst in an atmosphere. The method is simple to operate, mild in conditions, and the catalyst has high stability and good reusability. The production process of 2,5-furandicarboxylic acid is economical and environmentally friendly.

### CROSS-REFERENCE TO RELATED APPLICATIONS

This application claims priority to Chinese Patent Application No. 202510554914.0, filed on Apr. 29, 2025, which is hereby incorporated by reference in its entirety.

### TECHNICAL FIELD

The present disclosure relates to the field of chemical engineering technologies, and in particular, to a catalyst, preparation method and application for catalytic synthesis of 2,5-furandicarboxylic acid.

### BACKGROUND

Biomass is a widely existing renewable organic carbon resource in nature. Developing efficient catalytic conversion technologies to convert biomass resources into high-value chemicals is of great significance for achieving sustainable development. 5-hydroxymethylfurfural (HMF) is an important biobased platform molecule that can be synthesized through hydrolysis, isomerization, and dehydration reactions of cellulose and hemicellulose. The selective oxidation product of HMF, that is, 2,5-furandicarboxylic acid (FDCA), has a unique furan ring structure and dicarboxylic acid functional group, rendering it an important monomer for the synthesis of biobased polyethylene glycol to 2,5-furandicarboxylic acid (PEF). It is worth noting that PEF has similar structural characteristics to traditional petroleum-based polyethylene terephthalate (PET), as well as excellent biodegradability and gas barrier properties. It has passed the EU food safety certification, demonstrating broad application prospects in packaging materials and other fields. Therefore, designing an efficient catalytic process to achieve efficient and selective oxidation of biomass based raw material HMF for the preparation of FDCA has important research and application value. At present, the process of preparing HMF from biomass materials such as cellulose and glucose is relatively mature, which lays a solid foundation for establishing a new process for HMF catalytic oxidation synthesis of FDCA based on biomass route.

In traditional synthesis processes, HMF oxidation is mainly used to prepare FDCA through stoichiometric oxidation and homogeneous oxidation methods. The stoichiometric oxidation method usually uses strong oxidants such as KMnO₄, N₂O₄, HNO₃, etc.; However, such oxidants not only have strong corrosiveness to reaction equipment, but also generate many pollutants, which do not comply with the principles of green chemistry. Homogeneous catalytic method often uses Co²⁺/Mn²⁺/Br⁻ or Co²⁺/Zn²⁺/Br⁻ catalytic systems, with oxygen-containing gas as the oxidant; although this method avoids the use of stoichiometric oxidants, the yield of FDCA is generally low, and metal ions in the catalyst system are difficult to effectively recover. Bromine co catalysts are highly corrosive and can produce toxic brominated by-products. These technological bottlenecks have constrained the industrial application of traditional HMF oxidation processes.

Compared with stoichiometric oxidation and homogeneous catalysis, multiphase catalytic oxidation has significant advantages in product separation, catalyst recovery and utilization, catalytic efficiency, and environmental friendliness. Molecular sieve materials, as an ideal carrier for multiphase catalysts, can effectively disperse and stabilize metal active components through encapsulation and confinement, and can precisely regulate reaction selectivity and significantly improve catalytic performance. Therefore, the development of transition metal catalysts based on molecular sieve carriers is expected to provide an efficient and economical catalytic solution for HMF oxidation reactions. Organic functionalization modification can regulate the acid-base properties of molecular sieves and create suitable conditions for the loading and coating of metal active components. Based on this, constructing transition metal active centers through precise design of molecular sieve pore microenvironment, synthesizing catalysts with high stability and activity, and achieving highly selective catalytic oxidation of HMF has become a research hotspot in the field of catalysis and has received widespread attention.

### SUMMARY

According to a first aspect of the present disclosure, a method for preparing a catalyst for catalyzing a synthesis of 2,5-furandicarboxylic acid is provided, where a nitrogen-containing organic modified molecular sieve is used, followed by an introduction of a metal active component to obtain a nitrogen modified molecular sieve coated transition metal oxide catalyst. In the present disclosure, metal oxide active component in the catalyst structure is encapsulated within pores of the molecular sieve, forming uniformly dispersed nanostructures to enhance the stability and catalytic activity of the catalyst. This catalyst can efficiently catalyze the oxidation of 5-hydroxymethylfurfural to 2,5-furandicarboxylic acid under mild conditions in an oxygen-containing atmosphere.

A carrier of the transition metal oxide catalyst coated with molecular sieves is at least one of SBA-15, TS-1, ZSM-5, MCM-41, KIT-6, Beta, or HY molecular sieves; the active component of the transition metal oxide is at least one of iron oxide (FeₓO_{y}), cobalt oxide (CoₓO_{y}), copper oxide (CuₓO_{y}), manganese oxide (MnₓO_{y}), or vanadium oxide (VₓO_{y}), and from at least one of nitrate, sulfate, acetate, and acetylacetonate derived from transition metals of iron, cobalt, copper, manganese, and vanadium; the nitrogen modified structure of the catalyst is derived from at least one of 2,2′-bipyridine, 2,2′-bipyridineamine, tripyridine, phenanthroline, dicyandiamide, melamine, urea, triethylamine, ethylenediamine, diethylenetriamine, acrylamide, lysine, histidine, or C₃N₄.

The transition metal oxide component in the catalyst is uniformly dispersed and has nanometer sizes ranging from 5-50 nm; a loading amount of the metal component in the catalyst is 3.0 wt %-30.0 wt %.

The transition metal oxide catalyst coated with molecular sieve is obtained by impregnation-pyrolysis method or ball milling-pyrolysis method. The specific preparation steps are as follows: (1) adding a dealuminated molecular sieve to a solution of nitrogen-containing organic compound, heating and stirring, then removing the solvent, and conducting high-temperature pyrolysis in an inert gas atmosphere to obtain the nitrogen modified molecular sieve; (2) adding the nitrogen modified molecular sieve to a solution containing transition metal salts, subjecting to ultrasonic treatment, heating and stirring, loading metal component, then removing the solvent, and calcining in an air atmosphere using a muffle furnace to obtain the nitrogen modified molecular sieve coated transition metal oxide catalyst; or nitrogen modified molecular sieves directly mixing with transition metal salt, ball milling in a ball mill to evenly mix the two, and then calcining in an air atmosphere to obtain the nitrogen modified molecular sieve coated transition metal oxide catalyst.

A concentration of nitrogen-containing organic compound in step (1) is 0.2-2M; a mass ratio of nitrogen-containing organic compound to the molecular sieve is 0.1-1; a stirring temperature is 25-80 °C and a stirring time is 0.5-10 h; a pyrolysis temperature is 300-500 °C and a pyrolysis time is 1-5 h.

A concentration of the transition metal salt solution in step (2) is 0.01-0.1M; a mass ratio of the transition metal salt to a nitrogen doped molecular sieve is 0.1-0.5; a stirring temperature is 25-60 °C and a stirring time is 2-10 hours; a ball milling time is 2-10 hours and a temperature is room temperature; a calcination temperature is 400-800 °C and a calcination time is 1-5 hours.

The method for removing solvents in steps (1) and (2) involves rotary evaporation.

The above catalysts have mild synthesis conditions and are easy to operate. The catalysts obtained through the above technologies are low-cost, stable in performance, and have good reusability and recyclability.

A second aspect of the present disclosure provides an application method for the catalyst for a catalytic synthesis of 2,5-furandicarboxylic acid, including: contacting a nitrogen modified molecular sieve coated transition metal oxide catalyst with the raw material of 5-hydroxymethylfurfural in a kettle reactor or round bottom flask, and reacting and acidifying in the presence of an alkaline additive, water, and oxygen-containing atmosphere to obtain the 2,5-furandicarboxylic acid.

A molar concentration of 5-hydroxymethylfurfural is 0.5-1.5 mol/L.

The alkaline additive is at least one of LiOH, Li₂CO₃, CH₃COOLi, NaOH, Na₂CO₃, NaHCO₃, CH₃COONa, KOH, K₂CO₃, KHCO₃, CH₃COOK; a molar ratio of alkaline additive to 5-hydroxymethylfurfural is 1:1 to 4:1.

A molar ratio of the metal component of the catalyst to 5-hydroxymethylfurfural is 0.05:1 to 0.2:1.

The oxygen-containing atmosphere is oxygen or air, and a method of adding the oxygen-containing atmosphere includes introducing an oxygen-containing atmosphere into a reaction system, with a gas pressure of atmospheric pressure to 2.0 MPa; a bubbling method is used to introduce an oxygen-containing atmosphere with a gas flow rate of 5-60 mL/min.

A reaction temperature is 50-120 °C; and a reaction time is 5-12 hours.

An acidification process uses sulfuric acid or hydrochloric acid aqueous solution with a concentration of 0.1-1M and an acidification time of 5-30 minutes.

According to the present disclosure, the transition metal oxide catalyst coated with molecular sieve is used, and air or oxygen is used as the oxygen source. The reaction is carried out at a reaction temperature of 50-120 °C and a reaction pressure of atmospheric pressure to 2.0 MPa for 5-12 hours to efficiently and selectively catalyze the oxidation of 5-hydroxymethylfurfural to 2,5-furandicarboxylic acid. The conversion rate of 5-hydroxymethylfurfural and a yield of 2,5-furandicarboxylic acid both reach over 90%.

In the present disclosure, the catalyst prepared can efficiently and selectively catalyze the oxidation of 5-hydroxymethylfurfural to 2,5-furandicarboxylic acid in green solvent water using oxygen or air as the oxidant. The conversion rate of 5-hydroxymethylfurfural and the yield of 2,5-furandicarboxylic acid both reach over 90%. This method is easy to operate and has mild conditions. The product separation process is simple, efficient, and low-cost, and is a new method for preparing 2,5-furandicarboxylic acid based on biomass raw materials, with good application prospects.

The beneficial effects that the present disclosure can produce include the following.

(1) The catalyst prepared by the preparation method provided by the present disclosure encapsulates metal oxides in the pores of nitrogen modified molecular sieve, and the metal species exist in the form of nanoparticles, which have stability and high activity during the reaction process.

(2) The present disclosure uses the transition metal catalyst coated with nitrogen modified molecular sieve to achieve efficient catalytic oxidation of 5-hydroxymethylfurfural to 2,5-furandicarboxylic acid under mild conditions, energy-saving, and low product separation costs.

(3) The preparation method provided by the present disclosure has the advantages of low cost and low dosage of transition metal catalysts.

(4) The catalyst preparation method and the application method for synthesizing 2,5-furandicarboxylic acid provided by the present disclosure have innovation and strong promotion and application value.

### DESCRIPTION OF EMBODIMENTS

The present disclosure will be described below in combination with the embodiments, but the present disclosure is not limited to these embodiments.

Unless otherwise specified, the raw materials in the embodiments of the present disclosure are purchased through commercial channels.

In the embodiments of the present disclosure, a conversion rate of raw materials and a selectivity of product are calculated based on a molar amount of the substance, and the calculation formula is as follows:

raw material conversion rate=(addition amount of raw material−remaining amount of raw material)/addition amount of raw material*100% (1)

product selectivity=molar amount of product/molar amount of all products (2)

Example 1: Adding dealuminated SBA-15 molecular sieve (with a mass ratio of 0.5 between melamine to molecular sieve) to 50 mL of ethanol solution of melamine (concentration of 0.5M), stirring at 80 °C for 5 hours, removing the solvent by rotary evaporation, drying in an 80 °C oven for 12 hours, and heating resulting product in nitrogen at a heating rate of 10 °C/min and maintaining at 600 °C for 4 hours to obtain Me-SBA-15. Me represents that a nitrogen doped structure of SBA-15 molecular sieve is derived from melamine. Adding Me-SBA-15 (with a mass ratio of cobalt acetate to Me-SBA-15 being 0.2) to 50 mL of ethanol solution of cobalt acetate (concentration of 0.1M), sonicating thoroughly, then stirring at 60 °C for 5 hours, removing the solvent by rotary evaporation, and drying in an 80 °C oven for 12 hours. The resulting product being heated in air at a rate of 20 °C/min and maintained at 500 °C for 6 hours to obtain CoₓO_{y}/Me-SBA-15-WI catalyst (Co: 5.3 wt %). WI represents an introduction of the transition metal salt into the molecular sieve structure by impregnation method.

Adding CoₓO_{y}/Me-SBA-15-WI catalyst, 0.015 mol 5-hydroxymethylfurfural, 0.06 mol NaHCO₃, and 20 mL deionized water into a stainless-steel high-pressure reactor lined with polytetrafluoroethylene, where metal: 5-hydroxymethylfurfural=0.15:1 (mol:mol). Using an automatic temperature controller to raise a reaction temperature to 100 °C, adding 1.0 MPa oxygen, and reacting for 10 hours and keeping a pressure constant during a reaction process. After acidification of the reaction product, HPLC was used for analysis to obtain the conversion rate and product selectivity of reactants. The catalyst was recovered and reused for the catalytic oxidation of 5-hydroxymethylfurfural four times. After the reaction was completed, HPLC analysis was performed, and the reaction results are shown in Table 1.

Example 2: Adding dealuminated SBA-15 molecular sieve (with a mass ratio of 0.5 between dicyandiamide and molecular sieve) to 50 mL of ethanol solution of dicyandiamide (concentration of 0.5M), stirring at 80 °C for 5 hours, removing the solvent by rotary evaporation, drying in an 80 °C oven for 12 hours, and heating resulting product in nitrogen at a heating rate of 10 °C/min and maintaining at 600 °C for 4 hours to obtain DCD-SBA-15. DCD indicates that a nitrogen doped structure of SBA-15 molecular sieve is derived from dicyandiamide. Adding DCD-SBA-15 (mass ratio of cobalt acetate to DCD-SBA-15 is 0.2) to 50 mL of ethanol solution of cobalt acetate (concentration of 0.1M), sonicating thoroughly, then stirring at 60 °C for 5 hours, removing the solvent by rotary evaporation, and drying in an 80 °C oven for 12 hours. The resulting product is heated in air at a heating rate of 20 °C/min and maintained at 500 °C for 6 hours to obtain CoₓO_{y}/DCD-SBA-15-WI catalyst (Co: 4.8 wt %). WI represents an introduction of transition of metal salt into the molecular sieve structure by impregnation method.

Adding CoₓO_{y}/DCD-SBA-15-WI catalyst, 0.015 mol 5-hydroxymethylfurfural, 0.06 mol NaHCO₃, and 20 mL deionized water into a stainless-steel high-pressure reactor lined with polytetrafluoroethylene, where metal: 5-hydroxymethylfurfural=0.15:1 (mol:mol). Using an automatic temperature controller to raise a reaction temperature of 100 °C, adding 1.0 MPa oxygen, and reacting for 10 hours and keeping the pressure constant during the reaction process. After acidification of the reaction product, HPLC was used for analysis to obtain the conversion rate and product selectivity of reactants. The catalyst was recovered and reused for the catalytic oxidation of 5-hydroxymethylfurfural four times. After the reaction was completed, HPLC analysis was performed, and the reaction results are shown in Table 1.

Example 3: Adding dealuminated ZSM-5 molecular sieve (mass ratio of melamine to molecular sieve is 0.5) to 50 mL of ethanol solution of dicyandiamide (concentration is 0.5M), stirring at 80 °C for 5 hours, removing solvent by rotary evaporation, drying in an 80 °C oven for 12 hours, and heating resulting product in nitrogen at a heating rate of 10 °C/min, maintaining at 600 °C for 4 hours to obtain DCD-ZSM-5. DCD indicates that a nitrogen doped structure of ZSM-5 molecular sieve is derived from dicyandiamide. Adding DCD-ZSM-5 (mass ratio of cobalt acetate to DCD-ZSM-5 is 0.2) to 50 mL of cobalt acetate ethanol solution (concentration of 0.1M), sonicating thoroughly, then stirring at 60 °C for 5 hours, removing the solvent by rotary evaporation, and drying in an 80 °C oven for 12 hours. The resulting product is heated in air at a rate of 20 °C/min and maintained at 500 °C for 6 hours to obtain CoₓO_{y}/DCD-ZSM-5-WI catalyst (Co: 5.2 wt %), where WI represents transition metal salt introduced into the molecular sieve structure by impregnation.

Adding CoₓO_{y}/DCD-ZSM-5-WI catalyst, 0.015 mol 5-hydroxymethylfurfural, 0.06 mol NaHCO₃, and 20 mL deionized water into a stainless-steel high-pressure reactor lined with polytetrafluoroethylene, where metal: 5-hydroxymethylfurfural=0.15:1 (mol:mol). Using an automatic temperature controller to raise the reaction temperature to 100 °C, adding 1.0 MPa oxygen, and reacting for 10 hours and keeping the pressure constant during the reaction process. After acidification of the reaction product, HPLC was used for analysis to obtain the conversion rate and product selectivity of reactants. The catalyst was recovered and reused for the catalytic oxidation of 5-hydroxymethylfurfural four times. After the reaction was completed, HPLC analysis was performed, and the reaction results are shown in Table 1.

Example 4: Adding dealuminated HY molecular sieve (mass ratio of melamine to molecular sieve is 0.5) to 50 mL of ethanol solution of melamine (concentration is 0.5M), stirring at 80 °C for 5 hours, removing solvent by rotary evaporation, drying in an 80 °C oven for 12 hours, and heating the resulting product in nitrogen at a heating rate of 10 °C/min, maintaining at 600 °C for 4 hours to obtain DCD-HY DCD represents that the nitrogen doped structure of HY molecular sieve is derived from melamine. Adding DCD-HY (with a mass ratio of 0.2 between cobalt acetate to DCD-HY) to 50 mL of ethanol solution of cobalt acetate (concentration of 0.1M), sonicating thoroughly, then stirring at 60 °C for 5 hours, removing the solvent by rotary evaporation, and drying in an 80 °C oven for 12 hours. The resulting product is heated in air at a heating rate of 20 °C/min and maintained at 500 °C for 6 hours to obtain CoₓO_{y}/DCD-HY-WI catalyst (Co: 4.9 wt %). WI represents the introduction of transition metal salts into the molecular sieve structure by impregnation.

Adding CoₓO_{y}/DCD-HY-WI catalyst, 0.015 mol 5-hydroxymethylfurfural, 0.06 mol NaHCO₃, and 20 mL deionized water into a stainless-steel high-pressure reaction vessel lined with polytetrafluoroethylene, where metal: 5-hydroxymethylfurfural=0.15:1 (mol:mol). Using an automatic temperature controller to raise the reaction temperature to 100 °C, adding 1.0 MPa oxygen, and reacting for 10 hours and keeping a pressure constant during the reaction process. After acidification of the reaction product, HPLC was used for analysis to obtain the conversion rate and product selectivity of reactants. The catalyst was recovered and reused for the catalytic oxidation of 5-hydroxymethylfurfural four times. After the reaction was completed, HPLC analysis was performed, and the reaction results are shown in Table 1.

Example 5: Adding dealuminated SBA-15 molecular sieve (with a mass ratio of 0.5 between dicyandiamide and molecular sieve) to 50 mL of ethanol solution of dicyandiamide (concentration of 0.5M), stirring at 80 °C for 5 hours, removing the solvent by rotary evaporation, drying in an 80 °C oven for 12 hours, and heating the resulting product in nitrogen at a heating rate of 10 °C/min and maintaining at 600 °C for 4 hours to obtain DCD-SBA-15. DCD indicates that a nitrogen doped structure of SBA-15 molecular sieve is derived from dicyandiamide. Adding DCD-SBA-15 (mass ratio of manganese acetate to DCD-SBA-15 is 0.2) to 50 mL of ethanol solution of manganese acetate (concentration of 0.1M), sonicating thoroughly, then stirring at 60 °C for 5 hours, removing the solvent by rotary evaporation, and drying in an 80 °C oven for 12 hours. The resulting product is heated in air at a heating rate of 20 °C/min and maintained at 500 °C for 6 hours to obtain MnₓO_{y}/DCD-SBA-15-WI catalyst (Mn: 5.5 wt %). WI represents the introduction of transition metal salts into the molecular sieve structure by impregnation method.

Adding MnₓO_{y}/DCD-SBA-15-WI catalyst, 0.015 mol 5-hydroxymethylfurfural, 0.06 mol NaHCO₃, and 20 mL deionized water into a stainless-steel high-pressure reaction vessel lined with polytetrafluoroethylene, where metal: 5-hydroxymethylfurfural=0.15:1 (mol:mol). Using an automatic temperature controller to raise the reaction temperature of 100 °C, adding 1.0 MPa oxygen, and reacting for 10 hours and keeping the pressure constant during the reaction process. After acidification of the reaction product, HPLC was used for analysis to obtain the conversion rate and product selectivity of reactants. Retrieve the catalyst and reuse it for the catalytic oxidation of 5-hydroxymethylfurfural 4 times. After the reaction is complete, use HPLC analysis to obtain the conversion rate of reactants and product selectivity. The catalyst was recovered and reused for the catalytic oxidation of 5-hydroxymethylfurfural four times. After the reaction was completed, HPLC analysis was performed, and the reaction results are shown in Table 1.

Example 6: Adding dealuminated SBA-15 molecular sieve (with a mass ratio of 0.5 between dicyandiamide and molecular sieve) to 50 mL of ethanol solution of dicyandiamide (concentration of 0.5M), stirring at 80 °C for 5 hours, removing the solvent by rotary evaporation, drying in an 80 °C oven for 12 hours, and heating the resulting product in nitrogen at a heating rate of 10 °C/min and maintaining at 600 °C for 4 hours to obtain DCD-SBA-15. DCD indicates that a nitrogen doped structure of SBA-15 molecular sieve is derived from dicyandiamide. Adding DCD-SBA-15 (mass ratio of copper acetate to DCD-SBA-15 is 0.2) to 50 mL of ethanol solution of copper acetate (concentration of 0.1M), sonicating thoroughly, then stirring at 60 °C for 5 hours, removing the solvent by rotary evaporation, and drying in an 80 °C oven for 12 hours. The resulting product is heated in air at a heating rate of 20 °C/min and maintained at 500 °C for 6 hours to obtain CuₓO_{y}/DCD-SBA-15-WI catalyst (Cu: 4.6 wt %). WI represents the introduction of transition metal salts into the molecular sieve structure by impregnation method.

Adding CuₓO_{y}/DCD-SBA-15-WI catalyst, 0.015 mol 5-hydroxymethylfurfural, 0.06 mol NaHCO₃, and 20 mL deionized water into a stainless-steel high-pressure reaction vessel lined with polytetrafluoroethylene, where metal: 5-hydroxymethylfurfural=0.15:1 (mol:mol). Using an automatic temperature controller to raise the reaction temperature of 100 °C, adding 1.0 MPa oxygen, and reacting for 10 hours and keeping the pressure constant during the reaction process. After acidification of the reaction product, HPLC was used for analysis to obtain the conversion rate and product selectivity of reactants. The catalyst was recovered and reused for the catalytic oxidation of 5-hydroxymethylfurfural four times. After the reaction was completed, HPLC analysis was performed, and the reaction results are shown in Table 1.

Example 7: Adding dealuminated SBA-15 molecular sieve (with a mass ratio of 0.5 between dicyandiamide and molecular sieve) to 50 mL of ethanol solution of dicyandiamide (concentration of 0.5M), stirring at 80 °C for 5 hours, removing the solvent by rotary evaporation, drying in an 80 °C oven for 12 hours, and heating the resulting product in nitrogen at a heating rate of 10 °C/min and maintaining at 600 °C for 4 hours to obtain DCD-SBA-15. DCD indicates that a nitrogen doped structure of SBA-15 molecular sieve is derived from dicyandiamide. Adding DCD-SBA-15 (the mass ratio of cobalt acetate to DCD-SBA-15 is 0.1, and the mass ratio of copper acetate to DCD-SBA-15 is 0.1) to a mixed ethanol solution of 50 mL cobalt acetate and copper acetate (both concentrations of cobalt acetate and copper acetate are 0.05M), sonicating thoroughly, then stirring at 60 °C for 5 hours, removing the solvent by rotary evaporation, and drying in an 80 °C oven for 12 hours. The resulting product is heated in air at a heating rate of 20 °C/min and maintained at 500 °C for 6 hours to obtain CoₓCu_{y}O_{z}/DCD-SBA-15-WI catalyst (Co: 3.1 wt %, Cu: 2.1 wt %). WI represents the introduction of transition metal salts into the molecular sieve structure by impregnation method.

Adding CoₓCu_{y}O_{z}/DCD-SBA-15-WI catalyst, 0.015 mol 5-hydroxymethylfurfural, 0.06 mol NaHCO₃, and 20 mL deionized water into a stainless-steel high-pressure reaction vessel lined with polytetrafluoroethylene, where metal: 5-hydroxymethylfurfural=0.15:1 (mol:mol). Using an automatic temperature controller to raise the reaction temperature of 100 °C, adding 1.0 MPa oxygen, and reacting for 10 hours and keeping the pressure constant during the reaction process. After acidification of the reaction product, HPLC was used for analysis to obtain the conversion rate and product selectivity of reactants. The catalyst was recovered and reused for the catalytic oxidation of 5-hydroxymethylfurfural four times. After the reaction was completed, HPLC analysis was performed, and the reaction results are shown in Table 1.

Example 8: adding dealuminated SBA-15 molecular sieve (with a mass ratio of 0.5 between dicyandiamide and molecular sieve) to 50 mL of ethanol solution of dicyandiamide (concentration of 0.5M), stirring at 80 °C for 5 hours, removing the solvent by rotary evaporation, drying in an 80 °C oven for 12 hours, and heating the resulting product in nitrogen at a heating rate of 10 °C/min and maintaining at 600 °C for 4 hours to obtain DCD-SBA-15. DCD indicates that the nitrogen doped structure of SBA-15 molecular sieve is derived from dicyandiamide. Adding DCD-SBA-15 (the mass ratio of cobalt acetate to DCD-SBA-15 is 0.1, and the mass ratio of manganese acetate to DCD-SBA-15 is 0.1) to a mixed ethanol solution of 50 mL cobalt acetate and manganese acetate (both concentrations of cobalt acetate and manganese acetate are 0.05M), sonicating thoroughly, then stirring at 60 °C for 5 hours, removing the solvent by rotary evaporation, and drying in an 80 °C oven for 12 hours. The resulting product is heated in air at a heating rate of 20 °C/min and maintained at 500 °C for 6 hours to obtain CoₓMn_{y}O_{z}/DCD-SBA-15-WI catalyst (Co: 2.5 wt %, Mn: 2.6 wt %). WI represents the introduction of transition metal salts into the molecular sieve structure by impregnation method.

Adding CoₓMn_{y}O_{z}/DCD-SBA-15-WI catalyst, 0.015 mol 5-hydroxymethylfurfural, 0.06 mol NaHCO₃, and 20 mL deionized water into a stainless-steel high-pressure reaction vessel lined with polytetrafluoroethylene, where metal: 5-hydroxymethylfurfural=0.15:1 (mol:mol). Using an automatic temperature controller to raise the reaction temperature of 100 °C, adding 1.0 MPa oxygen, and reacting for 10 hours and keeping the pressure constant during the reaction process. After acidification of the reaction product, HPLC was used for analysis to obtain the conversion rate and product selectivity of reactants. The catalyst was recovered and reused for the catalytic oxidation of 5-hydroxymethylfurfural four times. After the reaction was completed, HPLC analysis was performed, and the reaction results are shown in Table 1.

Example 9: Adding dealuminated TS-1 molecular sieve (mass ratio of ortho phenanthroline to molecular sieve is 0.75) to 50 mL ethanol solution of ortho phenanthroline (concentration of 1.0M), stirring at 60 °C for 5 hours, removing solvent by rotary evaporation, drying in an 80 °C oven for 12 hours, and heating the resulting product in nitrogen at a heating rate of 20 °C/min, maintaining at 800 °C for 2 hours to obtain Phen-TS-1. Phen represents that the nitrogen doped structure of TS-1 molecular sieve is derived from ortho phenanthroline. Adding Phen-TS-1 (mass ratio of acetylacetonate vanadium oxide to Phen-TS-1 is 0.2) to a mixed ethanol solution of 50 mL acetylacetonate vanadium oxide (concentration of acetylacetonate vanadium oxide is 0.05M), sonicating thoroughly, then stirring at 80 °C for 5 hours, removing the solvent by rotary evaporation, drying in an 80 °C oven for 12 hours, and heating the resulting product in air at a heating rate of 20 °C/min, maintaining at 600 °C for 5 hours to obtain VₓO_{y}/Phen-TS-1-WI catalyst (V: 6.1 wt %). WI represents the introduction of transition metal salts into the molecular sieve structure by impregnation method.

Adding VₓO_{y}/Phen-TS-1-WI catalyst, 0.01 mol/5-hydroxymethylfurfural, 0.06 mol NaOH, and 20 mL deionized water into a three necked flask, where metal: 5-hydroxymethylfurfural=0.2:1 (mol:mol). Using an automatic temperature controller program to raise the temperature to 60 °C, oxygen was added to the reaction system at a rate of 40 mL/min using a bubbling method, and the reaction lasted for 12 hours. After acidification of the reaction product, HPLC was used for analysis to obtain the conversion rate and product selectivity of reactants. The catalyst was recovered and reused for the catalytic oxidation of 5-hydroxymethylfurfural four times. After the reaction was completed, HPLC analysis was performed, and the reaction results are shown in Table 1.

Example 10: Adding dealuminated HY molecular sieve (mass ratio of ortho phenanthroline to molecular sieve is 0.75) to 50 mL ethanol solution of ortho phenanthroline (concentration of 1.0M), stirring at 60 °C for 5 hours, removing solvent by rotary evaporation, drying in an 80 °C oven for 12 hours, and heating the resulting product in nitrogen at a heating rate of 20 °C/min, maintaining at 800 °C for 2 hours to obtain Phen HY, where Phen represents the nitrogen doped structure of HY molecular sieve derived from ortho phenanthroline. Adding Phen HY (mass ratio of acetylacetonate vanadium oxide to Phen HY is 0.2) to a mixed ethanol solution of 50 mL acetylacetonate vanadium oxide (concentration of acetylacetonate vanadium oxide is 0.05M), sonicating thoroughly, then stirring at 80 °C for 5 hours, removing the solvent by rotary evaporation, drying in an 80 °C oven for 12 hours, and heating the resulting product in air at a heating rate of 20 °C/min, maintaining at 600 °C for 5 hours to obtain VₓO_{y}/Phen HY WI catalyst (V: 5.1 wt %), where WI represents transition metal salts introduced into the molecular sieve structure by impregnation.

Adding VₓO_{y}/Phen HY-WI catalyst, 0.01 mol/5-hydroxymethylfurfural, 0.06 mol NaOH, and 20 mL deionized water into a three necked flask, where metal: 5-hydroxymethylfurfural=0.2:1 (mol:mol). Using an automatic temperature controller program to raise the temperature to 60 °C, oxygen was added to the reaction system at a rate of 40 mL/min using a bubbling method, and the reaction lasted for 12 hours. After acidification of the reaction product, HPLC was used for analysis to obtain the conversion rate and product selectivity of reactants. The catalyst was recovered and reused for the catalytic oxidation of 5-hydroxymethylfurfural four times. After the reaction was completed, HPLC analysis was performed, and the reaction results are shown in Table 1.

Example 11: Adding dealuminated ZSM-5 molecular sieve (with a mass ratio of C₃N₄ to molecular sieve of 0.5) to a 50 mL C₃N₄ ethanol solution (with a concentration of 0.5), stirring at 60 °C for 10 hours, removing the solvent by rotary evaporation, and drying in an 80 °C oven for 12 hours. The resulting product is heated in nitrogen at a rate of 10 °C/min and maintained at 700 °C for 2 hours to obtain C₃N₄-ZSM-5. C₃N₄ indicates that the nitrogen doped structure of ZSM-5 molecular sieve originates from C₃N₄. Mixing cobalt acetate, manganese acetate, and C₃N₄-ZSM-5 (the mass ratio of cobalt acetate to C₃N₄-ZSM-5 is 0.16, and the mass ratio of manganese acetate to C₃N₄-ZSM-5 is 0.04), then ball mill in a ball mill at a speed of 400 revolutions per minute for 6 hours. The resulting product is heated in air at a rate of 20 °C/min and maintained at 500 °C for 6 hours to obtain CoₓMn_{y}O_{z}/C₃N₄-ZSM-5-BM catalyst (Co: 3.9 wt %, Mn: 0.7 wt %). BM represents the introduction of transition metal salts into the molecular sieve structure by ball milling.

Adding CoₓMn_{y}O_{z}/C₃N₄-ZSM-5-BM catalyst, 0.015 mol 5-hydroxymethylfurfural, 0.03 mol Na₂CO₃, and 20 mL deionized water into a stainless-steel high-pressure reaction vessel lined with polytetrafluoroethylene, where metal: 5-hydroxymethylfurfural=0.15:1 (mol:mol). Using an automatic temperature controller program to raise the reaction temperature of 120 °C, adding 1.0 MPa oxygen, and reacting for 5 hours, keeping the pressure constant during the reaction process. After acidification of the reaction product, HPLC was used for analysis to obtain the conversion rate and product selectivity of reactants. The catalyst was recovered and reused for the catalytic oxidation of 5-hydroxymethylfurfural four times. After the reaction was completed, HPLC analysis was performed, and the reaction results are shown in Table 1.

Example 12: Adding dealuminated ZSM-5 molecular sieve (with a mass ratio of C₃N₄ to molecular sieve of 0.5) to a 50 mL C₃N₄ ethanol solution (with a concentration of 0.5), stirring at 60 °C for 10 hours, removing the solvent by rotary evaporation, and drying in an 80 °C oven for 12 hours. The resulting product is heated in nitrogen at a rate of 10 °C/min and maintained at 700 °C for 2 hours to obtain C₃N₄-ZSM-5. C₃N₄ indicates that the nitrogen doped structure of ZSM-5 molecular sieve originates from C₃N₄. Mixing cobalt acetate, copper acetate, and C₃N₄-ZSM-5 (the mass ratio of cobalt acetate to C₃N₄-ZSM-5 is 0.16, and the mass ratio of copper acetate to C₃N₄-ZSM-5 is 0.04), then ball mill in a ball mill at a speed of 400 revolutions per minute for 6 hours. The resulting product is heated in air at a rate of 20 °C/min and maintained at 500 °C for 6 hours to obtain CoₓCu_{y}O_{z}/C₃N₄-ZSM-5-BM catalyst (Co: 4.4 wt %, Cu: 1.2 wt %). BM represents the introduction of transition metal salts into the molecular sieve structure by ball milling.

Adding CoₓCu_{y}O_{z}/C₃N₄-ZSM-5-BM catalyst, 0.015 mol 5-hydroxymethylfurfural, 0.03 mol Na₂CO₃, and 20 mL deionized water into a stainless-steel high-pressure reaction vessel lined with polytetrafluoroethylene, where metal: 5-hydroxymethylfurfural=0.15:1 (mol:mol). Using an automatic temperature controller program to raise the reaction temperature to 120 °C, adding 1.0 MPa oxygen, and reacting for 5 hours, keeping a pressure constant during the reaction process. After acidification of the reaction product, HPLC was used for analysis to obtain the conversion rate and product selectivity of reactants. The catalyst was recovered and reused for the catalytic oxidation of 5-hydroxymethylfurfural four times. After the reaction was completed, HPLC analysis was performed, and the reaction results are shown in Table 1.

Example 13: Adding dealuminated KIT-6 molecular sieve (mass ratio of 2,2′-bipyridine to molecular sieve is 0.75) to a 50 mL ethanol solution of 2,2′-bipyridine (concentration is 0.5M), stirring at 80 °C for 5 hours, removing the solvent by rotary evaporation, drying in an 80 °C oven for 12 hours, and heating the resulting product in nitrogen at a heating rate of 20 °C/min. Maintaining at 500 °C for 4 hours to obtain Bpy-KIT-6. Bpy represents that the nitrogen doped structure of KIT-6 molecular sieve is derived from 2,2′-bipyridine. Adding Bpy-KIT-6 (a mass ratio of manganese nitrate to Bpy-KIT-6 is 0.4) to 50 mL of ethanol solution of manganese nitrate (concentration of 0.1M), sonicating thoroughly, then stirring at 80 °C for 5 hours, removing the solvent by rotary evaporation, and drying in an 80 °C oven for 12 hours. The resulting product is heated in air at a heating rate of 20 °C/min and maintained at 500 °C for 6 hours to obtain MnₓO_{y}/Bpy-KIT-6-WI catalyst (Mn: 7.8 wt %). WI represents the introduction of transition metal salts into the molecular sieve structure by impregnation method.

Adding MnₓO_{y}/Bpy-KIT-6-WI catalyst, 0.015 mol 5-hydroxymethylfurfural, 0.06 mol NaHCO₃, and 20 mL deionized water into a stainless-steel high-pressure reaction vessel lined with polytetrafluoroethylene, where metal: 5-hydroxymethylfurfural=0.15:1 (mol:mol). Using an automatic temperature controller program to raise the temperature to the reaction temperature of 120 °C, adding 1.0 MPa oxygen, and reacting for 12 hours, keeping the pressure constant during the reaction process. After acidification of the reaction product, HPLC was used for analysis to obtain the conversion rate and product selectivity of reactants. The catalyst was recovered and reused for the catalytic oxidation of 5-hydroxymethylfurfural four times. After the reaction was completed, HPLC analysis was performed, and the reaction results are shown in Table 1.

Example 14: Adding dealuminated KIT-6 molecular sieve (mass ratio of 2,2′-bipyridine to molecular sieve is 0.75) to a 50 mL ethanol solution of 2,2′-bipyridine (concentration is 0.5M), stirring at 80 °C for 5 hours, removing the solvent by rotary evaporation, drying in an 80 °C oven for 12 hours, and heating the resulting product in nitrogen at a heating rate of 20 °C/min. Maintaining at 500 °C for 4 hours to obtain Bpy-KIT-6. Bpy represents that the nitrogen doped structure of KIT-6 molecular sieve is derived from 2,2′-bipyridine. Mixing manganese nitrate with Bpy-KIT-6 (the mass ratio of manganese nitrate to Bpy-KIT-6 is 0.4), and then ball mill at a speed of 400 revolutions per minute for 6 hours in a ball mill. The resulting product is heated in air at a rate of 20 °C/min and maintained at 500 °C for 6 hours to obtain MnₓO_{y}/Bpy-KIT-6-BM catalyst (Mn: 8.3 wt %). BM represents transition metal salts introduced into the molecular sieve structure by ball milling.

Adding MnₓO_{y}/Bpy-KIT-6-BM catalyst, 0.015 mol 5-hydroxymethylfurfural, 0.06 mol NaHCO₃, and 20 mL deionized water into a stainless-steel high-pressure reaction vessel lined with polytetrafluoroethylene, where metal: 5-hydroxymethylfurfural=0.15:1 (mol:mol). Using an automatic temperature controller program to raise the temperature to the reaction temperature of 120 TC, adding 1.0 MPa oxygen, and reacting for 12 hours, keeping the pressure constant during the reaction process. After acidification of the reaction product, HPLC was used for analysis to obtain the conversion rate and product selectivity of reactants. The catalyst was recovered and reused for the catalytic oxidation of 5-hydroxymethylfurfural four times. After the reaction was completed, HPLC analysis was performed, and the reaction results are shown in Table 1.

TABLE 1
Catalytic oxidation results of 5-hydroxymethylfurfural on different catalysts

| | | Oxygen | | | | | | |
|---|---|---|---|---|---|---|---|---|
| | | pressure | | | HMFcon | | | |
| | | (Mpa)Or | Temperature | Time | version | Selectivity (%) | | |
| Example | Catalyst | flow rate | (°C) | (h) | rate (%) | FDCA | FFCA | Other |
| Example 1 | CoₓO_{y}/Me-SBA- | 1.0 | 100 | 10 | 92 | 71 | 17 | 12 |
| Example 1 | 15-WI | | | | 87 | 73 | 10 | 17 |
| reuse | | | | | | | | |
| Example 2 | CoₓO_{y}/DCD- | 1.0 | 100 | 10 | 95 | 89 | 4 | 7 |
| Example 2 | SBA-15-WI | | | | 91 | 90 | 2 | 8 |
| reuse | | | | | | | | |
| Example 3 | CoₓO_{y}/DCD- | 1.0 | 100 | 10 | >99 | 62 | 21 | 17 |
| Example 3 | ZSM-5-WI | | | | >99 | 51 | 26 | 23 |
| reuse | | | | | | | | |
| Example 4 | CoₓO_{y}/DCD- | 1.0 | 100 | 10 | >99 | 81 | 9 | 10 |
| Example 4 | HY-WI | | | | 92 | 79 | 11 | 10 |
| reuse | | | | | | | | |
| Example 5 | MnₓO_{y}/DCD- | 1.0 | 100 | 10 | 82 | 82 | 11 | 7 |
| Example 5 | SBA-15-WI | | | | 81 | 84 | 12 | 4 |
| reuse | | | | | | | | |
| Example 6 | CuₓO_{y}/DCD- | 1.0 | 100 | 10 | 73 | 21 | 52 | 27 |
| Example 6 | SBA-15-WI | | | | 67 | 26 | 41 | 33 |
| reuse | | | | | | | | |
| Example 7 | CoₓCu_{y}O_{z}/DCD- | 1.0 | 100 | 10 | >99 | 91 | 2 | 7 |
| Example 7 | SBA-15-WI | | | | 89 | 93 | 1 | 6 |
| reuse | | | | | | | | |
| Example 8 | CoₓMn_{y}O_{z}/DCD- | 1.0 | 100 | 10 | >99 | 93 | 4 | 3 |
| Example 8 | SBA-15-WI | | | | 95 | 88 | 8 | 4 |
| reuse | | | | | | | | |
| Example 9 | VₓO_{y}/Phen- | 40 mL/min | 60 | 12 | 79 | 33 | 51 | 16 |
| Example 9 | TS-1-WI | | | | 62 | 39 | 37 | 24 |
| reuse | | | | | | | | |
| Example 10 | VₓO_{y}/Phen- | 40 mL/min | 60 | 12 | 98 | 46 | 40 | 14 |
| Example 10 | HY-WI | | | | 81 | 54 | 27 | 19 |
| reuse | | | | | | | | |
| Example 11 | CoₓMn_{y}O_{z}/C₃N₄- | 1.0 | 120 | 5 | >99 | 72 | 15 | 13 |
| Example 11 | ZSM-5-BM | | | | >99 | 73 | 11 | 16 |
| reuse | | | | | | | | |
| Example 12 | CoₓCu_{y}O_{z}/C₃N₄- | 1.0 | 120 | 5 | 96 | 82 | 11 | 7 |
| Example 12 | ZSM-5-BM | | | | 97 | 79 | 12 | 8 |
| reuse | | | | | | | | |
| Example 13 | MnₓO_{y}/Bpy- | 1.0 | 120 | 12 | 97 | 89 | 6 | 5 |
| Example 13 | KIT-6-WI | | | | >99 | 82 | 10 | 8 |
| reuse | | | | | | | | |
| Example 14 | MnₓO_{y}/Bpy- | 1.0 | 120 | 12 | >99 | 74 | 8 | 18 |
| Example 14 | KIT-6-BM | | | | >99 | 68 | 6 | 26 |
| reuse | | | | | | | | |

HMF: 5-hydroxymethylfurfural; FFCA: 5-formyl-2-furancarboxylic acid; FDCA: 2,5-furandicarboxylic acid.

What is claimed is:

1. A catalyst for catalyzing a synthesis of 2,5-furandicarboxylic acid, wherein the catalyst is a molecular sieve coated transition metal oxide catalyst, and active component of the catalyst is a transition metal oxide that is selected from at least one of iron oxide, cobalt oxide, copper oxide, manganese oxide, or vanadium oxide, the active component is selected from at least one of nitrate, sulfate, acetate, and acetylacetonate of transition metals of iron, cobalt, copper, manganese, and vanadium;
a carrier of the catalyst is a nitrogen species modified molecular sieve, and the molecular sieve is selected from at least one of SBA-15, TS-1, ZSM-5, MCM-41, KIT-6, Beta, or HY molecular sieves;
the nitrogen species in a catalyst carrier structure is derived from nitrogen-containing organic compound, and the nitrogen-containing organic compound is selected from at least one of 2,2′-bipyridine, 2,2′-bipyridineamine, tripyridine, phenanthroline, dicyandiamide, melamine, urea, triethylamine, ethylenediamine, diethylenetriamine, acrylamide, lysine, histidine, or C₃N₄.

2. A method for preparing the catalyst for catalyzing a synthesis of 2,5-furandicarboxylic acid according to claim 1, wherein a method for preparing the nitrogen species modified molecular sieve comprises: adding the molecular sieve to an ethanol solution containing nitrogen-containing organic compound for impregnation reaction, then removing the solvent, and pyrolyzing in an inert atmosphere to obtain the nitrogen species modified molecular sieve.

3. The method for preparing the catalyst for catalyzing a synthesis of 2,5-furandicarboxylic acid according to claim 2, wherein an impregnation method of nitrogen-containing organic compound is heating and stirring impregnation, with a temperature of 25-80 °C and a time of 0.5-10 h; a concentration of the nitrogen-containing organic compound is 0.2-2M, and a mass ratio of nitrogen-containing species to molecular sieve is 0.1-1; after immersion, a pyrolysis temperature is 300-500 °C, a heating rate is 5-10 °C/min, and a pyrolysis time is 1-5 hours.

4. The method for preparing the catalyst for catalyzing a synthesis of 2,5-furandicarboxylic acid according to claim 2, wherein a preparation method of nitrogen modified molecular sieve coated metal oxide catalyst is as follows: adding nitrogen species modified molecular sieve to an ethanol solution containing transition metal salt, performing impregnation reaction, then removing the solvent, and calcining in an air atmosphere to obtain the nitrogen modified molecular sieve coated transition metal oxide catalyst; or mixing nitrogen modified molecular sieves with transition metal salt through solid grinding, and then calcining in an air atmosphere to obtain the nitrogen modified molecular sieve coated transition metal oxide catalyst.

5. The method for preparing the catalyst for catalyzing a synthesis of 2,5-furandicarboxylic acid according to claim 4, wherein an impregnation method of transition metal salt is heating and stirring impregnation, with a stirring temperature of 25-60 °C and a stirring time of 2-10 hours; a concentration of transition metal salt solution is 0.01-0.1M, and a mass ratio of transition metal salt to nitrogen doped molecular sieve is 0.1-0.5; after impregnation, a calcination temperature is 400-800 °C, a heating rate is 5-10 °C/min, a calcination time is 1-5 hours, and a calcination atmosphere is selected from at least one of nitrogen or argon gas, the nitrogen modified molecular sieve and transition metal salt are mixed by mechanical grinding in a ball mill, with a rotation speed of 200-800 revolutions per minute and a ball milling time of 2-10 hours.

6. The method for preparing the catalyst for catalyzing a synthesis of 2,5-furandicarboxylic acid according to claim 2, wherein a loading amount of the metal component in the nitrogen modified molecular sieve coated metal oxide catalyst is 3.0 wt %-30.0 wt %, and a content of nitrogen species is 2.0 wt %-20.0 wt %.

7. An application of the catalyst for catalyzing a synthesis of 2,5-furandicarboxylic acid according to claim 1, wherein 5-hydroxymethylfurfural is reacted with the catalyst in an oxygen-containing atmosphere to prepare 2,5-furandicarboxylic acid.

8. The application of the catalyst for catalyzing a synthesis of 2,5-furandicarboxylic acid according to claim 7, wherein it comprises the following steps: mixing the catalyst, 5-hydroxymethylfurfural, alkaline additive, and water, and reacting them in an oxygen-containing atmosphere; a reaction temperature is 50-120 °C, a reaction time is 5-12 hours, and a reaction pressure is atmospheric pressure of 2.0 MPa; after the reaction is complete, adding acid to obtain 2,5-furandicarboxylic acid.

9. The application of the catalyst for catalyzing a synthesis of 2,5-furandicarboxylic acid according to claim 8, wherein the alkaline additive is selected at least one from a group consisting of LiOH, Li₂CO₃, CH₃COOLi, NaOH, Na₂CO₃, NaHCO₃, CH₃COONa, KOH, K₂CO₃, KHCO₃, CH₃COOK; a molar ratio of the alkaline additive to 5-hydroxymethylfurfural is 1:1 to 4:1; a molar ratio of the metal component in the catalyst to 5-hydroxymethylfurfural is 0.05:1 to 0.2:1.
